(19) United States
(12) Patent Application Publication
(10) Pub. No.: US 2006/0188867 A1
(43) Pub. Date: Aug. 24, 2006

(54) Method of cryopreserving cells and tissues by liposomal delivery of sugars to enhance post-thaw viability

(75) Inventors: Jason P. Acker, Edmonton (CA); Maria Gyongyossy-Issa, Vancouver (CA)

Correspondence Address: OGILVY RENAULT LLP, 1981 MCGILL COLLEGE AVENUE, SUITE 1600, MONTREAL, QC, H3A2Y3, CA

(73) Assignee: Canadian Blood Services, Ottawa (CA)

(21) Appl. No.: 11/340,483

(22) Filed: Jan. 27, 2006

Related U.S. Application Data
(60) Provisional application No. 60/647,403, filed on Jan. 28, 2005.

(51) Int. Cl. A01N 1/02; C12N 5/08

(52) U.S. Cl. 435/2; 435/372

(57) ABSTRACT

A method for cryopreserving cells entails the liposomal delivery of intracellular sugar(s), such as trehalose, sucrose, raffinose, stachyose, and combinations thereof, into cells and tissues, such as red blood cells, for enhancing post-thaw viability. This method enables rapid and easy delivery of protective molecules into cells which thus greatly simplifies the preparation of cells for cryopreservation. Furthermore, as much lower concentrations of intracellular protectant are used, the method allows red blood cells containing the liposomally-delivered intracellular sugar to be transfused into a patient immediately following the thaw without having to first remove any of the cryoprotectant sugar.

### CROSS-REFERENCE TO RELATED APPLICATIONS

This application for patent claims priority from U.S. Provisional Patent Application Ser. No. 60/647,403 entitled METHOD FOR CRYOPRESERVATION OF CELLS AND TISSUES filed Jan. 28, 2005.

### TECHNICAL FIELD

The present invention relates generally to cryopreservation of cells and tissues and, in particular, to techniques for improving post-thaw viability of cryopreserved cells and tissues.

### BACKGROUND OF THE INVENTION

As is widely appreciated, cryopreservation of cells and tissues is vital to many different aspects of medicine and medical research such as the cryopreservation of blood supplies in blood banks and the cryopreservation of spermatozoa and embryos in fertility clinics. For the emerging fields of tissue engineering, cell and tissue transplantation and genetic technologies, preserving the functional viability of the native and induced characteristics of cells remains one of the most important challenges facing reparative medicine. Although cryopreservation is used in a wide range of applications, post-thaw viability remains a significant challenge. In the following paragraphs, the references believed to be most relevant to the subject matter of the present application are canvassed.

U.S. Pat. No. 6,770,478 (Crowe et al.), entitled “Erythrocytic Cells and Method for Preserving Cells”, describes a method of removing alcohol from erythrocytic cells, while loading the cells with oligosaccharide (e.g. trehalose) in order to preserve biological properties during freeze-drying and rehydration.

U.S. Pat. No. 6,713,245 (Koopmans et al.) describes a method for cryopreserving neural cells in a protein-free buffer containing glucose.

U.S. Pat. No. 6,673,607 (Toner et al.) describes a method for micro-injecting sugars into cells to improve viability following cryopreservation.

U.S. Pat. No. 6,610,531 (Mateczun et al.) describes a method of preserving live bacteria using extracellular trehalose.

U.S. Pat. No. 6,582,696 (Kuri-Harcuch et al.) describes various methods and products relating to the preservation of mammalian epithelial or mesenchymal cells. This method uses extracellular sugar for the preservation of cells used in artificial skins.

U.S. Pat. No. 5,827,741 (Beattie et al.) describes a method for loading trehalose into mammalian cells by altering the thermal conditions of the cells.

U.S. Pat. No. 5,827,640 (Wiggins et al.) describes the use of extracellular trehalose for the cryopreservation of cells.

U.S. Pat. No. 5,750,330 (Tometsko et al.) describes a method for lyophilizing red blood cells using extracellular trehalose.

U.S. Pat. No. 5,629,145 (Meryman et al.) describes a method of cryopreserving cell suspensions and red blood cells using extracellular sugar.

U.S. Pat. No. 5,242,792 (Rudolph et al.) describes the use of glycerol to permeabilize red blood cells and load sugar into red blood cells to improve the recovery of cells following freeze-drying.

PCT Application WO2004/011616 (Toner et al.) describes the use of intracellular sugars for the dry storage of cells and tissues. This application describes a pore-forming agent (a bacterial toxin) and a process of electroporation for loading sugars.

U.S. Pat. No. 6,127,177 (Toner et al.) describes a preservation method for biological materials, involving reversibly porating the cell membranes to enable intracellular loading of a sugar-containing bio-prevention agent.

U.S. Pat. No. 5,827,741 (Beattie et al.) describes the combination of trehalose and dimethyl sulfoxide as an effective cryoprotectant for cell clusters as well as platelets.

Choi et al. (“The Influence of cooling rate, developmental stage, and the addition of sugar on the cryopreservation of larvae of the pearl oyster Pinctada fucata martesii” in Cryobiology 46(2): 190-3 (2003)) report that the addition of sugar (0.2M glucose or sucrose) to a freezing medium including 2.0M dimethyl sulfoxide improves the survival rate of trochophore larvae following cryopreservation, and suggests a preferred cooling rate of 1 degree C./min for pearl oyster larvae.

Sasnoor et al. (“A combination of catalase and trehalose as additives to conventional freezing medium results in improved cryoprotection of human hematopoietic cells with reference to in vitro migration and adhesion properties” in Transfusion 45(4): 622-33 (2005)) report that the use of extracellular catalase and trehalose (i.e. in conventional freezing medium) affords improved preservation of adhesion- and migration-related properties on frozen cells.

Patist et al. (“Preservation mechanisms of trehalose in food and biosystems” in Collids Surf B Biointerfaces 40(2): 107-13 (2005)) describe the use of trehalose as a preservative. The use of trehalose as a non-toxic cryoprotectant in vaccines and organs for surgical transplant is suggested.

Elliott et al. (“Rapid loading of trehalose induced in J774 mouse macrophage cells” in Cryobiology 47: 257 (2003)) describe an ATP-dependent means of loading trehalose into cells to enhance the desiccation tolerance.

Russo et al. (“Reversible permeabilization of plasma membranes with an engineered switchable pore” in Nat Biotechnol. 15(3): 378-383, 1997) teach the reversible permeabilization and loading of sugars into cells using a bacterial toxin.

Satpathy et al. (“Loading red blood cells with trehalose: a step towards biostabilization” in Cryobiology 49 (2): 123-136, 2004) teach thermal poration of red blood cells and the loading of sugars for use in cryopreservation. In particular, it is suggested that the loading of trehalose into red blood cells is not only due to diffusion but is a combination of diffusion and thermotropic transition of the membrane lipids. As such, this reference discloses the concept of the intracellular loading of sugars, for the purpose of cell preservation.

Shulkin et al. (“Lyophilized liposomes: a new method for long-term vesicular storage”, J Microencapsul. 1984; 1(1): 73-80) describe the use of sugars for the preservation of liposomes during lyophilization and cryopreservation of liposomes.

Canadian Patent Application 2,379,366 (Abra et al.), entitled “A Liposome Composition having Resistance to Freeze/Thaw Damage”, teaches the use of sugars to protect liposomes from freeze/thaw damage. This patent application is directed to the protection of liposomes themselves.

U.S. Pat. No. 6,623,671 (Coe et al.) describes a method of sizing liposomes by passing a suspension of liposomes through an aluminum oxide porous film.

U.S. Pat. No. 6,372,720 (Longmuir et al.) describes liposome complexes and individual components thereof for intracellular and/or intranuclear delivery of substances. However, the substances delivered by the liposomes appear to be restricted to genes, RNA, oligonucleotides, antisense molecules, ribozymes, peptides, factors and various regulators and therapeutics.

U.S. Pat. No. 6,245,427 (Duzgunes et al.) describes a method of intracellular delivery and transfection of DNA, RNA, polypeptides, genes, proteins, drugs and biologically active agents into cells in vitro and in vivo. The vehicle comprises a mixture of a liposome and a polypeptide lacking specificity for cellular receptors. Therefore, this technology involves protein-mediated liposome fusion.

U.S. Pat. No. 5,292,524 (Male et al.) describes the loading of radiolabelled molecules into platelets using liposomes. This patent is directed to the use of liposomes as a vector for diagnostic and therapeutic agents.

U.S. Pat. No. 4,927,637 (Morano et al.) describes a method of sizing liposomes by extrusion.

Seville et al. (“Preparation of dry powder dispersions for non-viral gene delivery by freeze-drying and spray-drying” in J Gene Med. 2002; 4(4): 428-37) describe the use of freeze-dried liposomes to deliver genetic material into cells.

Ulrich (“Biophysical aspects of using liposomes as delivery vehicles” in Biosci Rep. 2002; 22(2): 129-50) provides a general summary of the use of liposomes as delivery vehicles.

U.S. Pat. No. 6,323,001 (Londesborough et al.) describes a method of encoding polypeptides found in yeast trehalose synthase for insertion by suitable vectors to transform host cells so that these host cells have an increased tolerance to dehydration.

U.S. Patent Application Publication 2005/0084481 (Hand et al.) describes the use of extracellular ATP in a concentration sufficient to open pores in the plasma membrane in order to stabilize a red blood cell for cryopreservation.

U.S. Pat. No. 5,425,951 (Goodrich et al.) describes a method of reconstituting lyopholized cells.

U.S. Pat. No. 5,340,592 (Goodrich et al.) describes a method of lyopholizing erythrocytes.

PCT Published Application WO99/15010 (Buhr et al.), entitled “Reduction of Sperm Sensitivity to Chilling”, teaches the incubation of spermatozoa with liposomes made of selected lipids and head plasma membranes to improve freeze/thaw viability.

Canadian Published Patent Application 2,128,527 (Goodrich et al.), entitled “Method of Freezing Cells and Cell-like Materials”, teaches a buffer containing a cryoprotectant (including a sugar) to stabilize intravesicle components, e.g. for use with membrane systems, including liposomes.

Pugh et al (“Cryopreservation of in vitro-produced bovine embryos: effects of protein type and concentration during freezing of liposomes during culture on post-thaw survival” in Theriogenology 50(3): 495-506 (1998)) disclose supplementing an in-vitro culture medium with liposomes containing lecithin, spingomyelin and cholesterol. The presence of the liposome compositions did not affect embryo development, and in the case of the lecithin composition significantly reduced survival after freezing. It was further suggested that the lecithin liposome preparations may affect the post-thaw embryo survival by altering embryonic membrane composition.

U.S. Pat. No. 5,800,978, U.S. Pat. No. 5,958,670, and U.S. Pat. No. 6,007,978, each of which is entitled “Method of Freezing Cells and Cell-like Materials” (Goodrich et al) describe cryoprotectants made of polyalcohols, saccharides and mixtures thereof.

European Patent EP0967862B1 to Menys et al. describes the use of intracellular trehalose (5-150 mM) to preserve human platelets during drying. The patent describes the means of delivering intracellular trehalose as being one of electropermeabilization, phase transition of the membrane, osmotic methods such as the use of organic osmolytes and pinocytosis, transient lysis methods such as acid shock and reversible cross-linking and the use of membrane permeable, esterase-labile trehalose derivatives.

PCT Published Patent Application W086/03938 to Crowe et al. describes the use of a preserving agent having at least two monosaccharide units used either internally or externally to preserve liposomes during freeze-drying.

European Patent EP0594651B1 to Aspinall describes the use of extracellular sugars and osmolytes to protect red blood cells during drying.

European Patent EP1221835B1 (PCT/GB00/04078; WO01/030141) to Stienstra describes the use of shear stress to pre-activate platelets and load trehalose inside for the purpose of improving platelet stabilization during drying.

While the foregoing references teach a number of useful techniques for cryopreservation, it is still widely accepted by those of ordinary skill in the art that the ability to reliably thaw cryopreserved cells and tissues remains a fundamental challenge. Therefore, methods and techniques that improve post-thaw viability of cryopreserved cells and tissues remain highly desirable.

### SUMMARY OF THE INVENTION

This invention provides a method for the cryopreservation of cells that maintains cell viability by the use of liposome-delivered intracellular sugars. Liposomes, composed of phospholipids and cholesterol, fuse with the cell plasma membrane and deliver a sugar or combination of sugars into the cell. Fusion of the liposomes with the cell membrane and the loading of intracellular sugars is shown to protect cells during cooling to, storage at, and warming from low temperatures. Control of the cooling and warming rates as well as the temperature of extracellular ice nucleation will enhance the effect of liposomal-delivered intracellular sugars on post-thaw cell viability. Liposomal-delivery of intracellular sugars provides suitable cellular protection and stabilization to permit long-term banking of cells for use in emerging cell-based technologies and therapies.

The use of liposome-delivered intracellular sugars for the cryopreservation of cells and tissues has many advantages over current glycerol- and DMSO-based techniques that could significantly alleviate the problems currently facing the long-term storage of biological material. Firstly, the liposomal delivery of intracellular sugar is a rapid, easy and simple process that expedites and simplifies cell preparation prior to cryopreservation. Secondly, the toxicity of sugars is significantly less than that of traditional cryoprotectants due to the fact that lower concentrations are required for protection. In addition, the presence of intracellular sugars has been shown to reduce the sensitivity of cells to cooling-rate specific injury, potentially eliminating the dependence on expensive controlled-rate freezers and decreasing the overall processing time. Finally, the low concentration of these natural cryoprotectants eliminates the need for a post-thaw processing step, allowing for products to be transfused or transplanted immediately after thawing.

Using liposomes to deliver sugars into cells is a significant improvement over current techniques for reversible permeabilization of cells. Electroporation and thermal shock require dedicated instrumentation and precise control of physical parameters that may be difficult to achieve for large samples. Chemical permeabilization using detergents or bioactive agents (bacterial toxins) and/or genetic manipulation may face potential immunogenic problems as well as regulatory barriers. Liposomal delivery does not require specialized instrumentation and liposomes are extremely biocompatible and FDA-approved for drug delivery. Accordingly, the present invention provides a methodology for cryopreservation of cells and tissues that is convenient, efficient and effective.

Therefore, in accordance with one aspect of the present invention, a method is provided for cryopreserving cells to enhance post-thaw cell viability. The method includes the steps of loading sugar into liposomes; fusing the liposomes to cell membranes of cells that are to be cryopreserved, thereby delivering the sugar into the cells as a cryoprotectant; cooling the cells to a predetermined nucleation temperature; nucleating extracellular ice; and cooling the cells to a temperature lower than the predetermined nucleation temperature.

The cells can be red blood cells, platelets in suspension, stem cells or other human or mammalian cells, tissues or organs.

In one embodiment, the sugar is selected from the group consisting of trehalose, sucrose, raffinose, and stachyose.

In another embodiment, the method includes a prior step of manufacturing the liposomes to contain: dipalmitoyl phosphatidylserine, dipalmitoyl phosphatidylcholine, and cholesterol.

In accordance with another aspect of the present invention, a method is provided for enhancing post-thaw viability of a cell to be cryopreserved. The method includes steps of: loading a cryoprotectant sugar into a liposome; and causing the liposome to fuse with the cell that is to be cryopreserved, the liposome delivering the cryoprotectant sugar into the cell for enhancing post-thaw viability of the cell.

In one embodiment, the method further includes a step of cryopreserving the cell containing the cryoprotectant sugar by cooling the cell below a nucleation temperature.

In another embodiment, the method further includes a subsequent step of thawing the cell by warming the cell above the nucleation temperature.

In yet another embodiment, the sugar is selected from the group consisting of trehalose., sucrose, raffinose, and stachyose.

### BRIEF DESCRIPTION OF THE DRAWINGS

Further features and advantages of the present invention will become apparent from the following detailed description, taken in combination with the appended drawings, in which:

FIG. 1A is a population distribution (“dot plot”) plotting the log of forward scatter (FS) versus the log of side scatter (SS) to differentially analyze RBCs and liposomes in a sample;

FIG. 1B is a flow cytometry plot of fluorescence events versus FITC, demonstrating that a 0.1 mM carboxyfluorescein (CF) contained within liposomes can be resolved;

FIG. 1C is a flow cytometry plot of fluorescence events versus FITC, demonstrating that RBCs in CF-containing media are not fluorescent since CF cannot cross into the RBCs;

FIG. 1D is a flow cytometry plot of fluorescence events versus FITC, demonstrating that CF and trehalose loaded into liposomes and mixed with red blood cells move from the liposomes into the red blood cells after overnight storage at 22° C.;

FIG. 1E is a flow cytometry plot of fluorescence events versus FITC, demonstrating that an increase of the liposome/RBC ratio results in an increase of fluorescence in the red blood cells, indicative of augmented delivery of sugar into the cells;

FIG. 2 is a bar graph plotting the recovery from 0° C. and from −40° C. of (i) red blood cells, (ii) red blood cells exposed to liposomes but containing no sugar and (iii) red blood cells exposed to liposomes containing 0.29M trehalose, as compared to 100% recovery from 22° C. (the control); and

FIG. 3 is a bar graph plotting the recovery from 22° C., 0° C., −5° C., −10° C., −20° C., −30° C. and −40° C. of (i) human platelets in buffer, (ii) human platelets in buffer exposed to liposomes without sugar, and (iii) human platelets in buffer exposed to liposomes containing 0.29M trehalose.

It will be noted that throughout the appended drawings, like features are identified by like reference numerals.)

### DESCRIPTION OF PREFERRED EMBODIMENTS

In accordance with a preferred embodiment of the present invention, a method for enhancing post-thaw viability of cryopreserved cells and tissues includes the step of loading sugar into liposomes (or “liposomal vesicles”). The sugar-containing liposomes serve as delivery vehicles for delivering cryoprotectant sugar(s) into cells that are to be cryopreserved. This method of cryopreserving cells enhances post-thaw cell viability.

In accordance with a preferred embodiment, the method includes the steps of (i) loading sugar into liposomes; (ii) fusing the liposomes to cell membranes of cells that are to be cryopreserved, thereby delivering the sugar into the cells as a cryoprotectant; (iii) cooling the cells to a predetermined nucleation temperature; (iv) nucleating extracellular ice; and (v) cooling the cells to a temperature lower than the predetermined nucleation temperature.

This method can be utilized to cryopreserve cells, tissues or other biological materials. Specifically, the method can be used to cryopreserve mammalian cells, such as human or other animal cells. The method can be used to cryopreserve blood cells, such as human red blood cells (erythrocytes) e.g. for cryopreservation of donated blood in blood banks. Likewise, the method can be used to cryopreserve platelets in a suspension or “buffer”. Furthermore, the method can be used to cryopreserve stem cells (multi-potent progenitor cells), components of tissues or organs, or other biological materials.

Preferably, the sugar utilized for liposomal delivery into the cells is selected from the group consisting of trehalose, sucrose, raffinose, and stachyose. Persons of ordinary skill in the art will, of course, appreciate that other types of sugars could be substituted where appropriate. Preferably, the sugar has a concentration between 0.05M and 0.50M. In the best mode known to the Applicant, trehalose is used in a concentration of 0.29M, although it should be understood that other sugars in similar or different concentrations can be used to work the present invention. A concentration of 0.29M is preferred because this concentration is iso-osmotic with normal saline and normal solutions that are typically used for cell suspensions. The concentration of trehalose (TRE) can be varied provided there is salt present to offset a lower TRE concentration. While the range of 0.05M-0.50M mentioned above is generally applicable to most monosaccharides and disaccharides, the optimal concentration for a given sugar will vary with a number of factors such as the molecular weight of the sugar. For example, raffinose may be protective at 100 mM versus the 200 mM commonly used for trehalose.

It should be understood that “sugar” or “sugar(s)” should be interpreted as including either a single type of sugar, e.g. trehalose, or a combination of sugars, such as for example a combination of sugars selected from the group consisting of trehalose, sucrose, raffinose, and stachyose.

Furthermore, in order to further enhance post-thaw viability, it is preferable that the sugar(s) be dissolved in a solution having a physiologic osmolality, conductivity and pH.

In accordance with a preferred embodiment of the present invention, the sugar (once liposomally delivered into the cells) should result in a concentration of intracellular sugar that is below a toxicity threshold both in terms of the loaded cells themselves or in terms of systemic toxicity, e.g. a blood transfusion of thawed cells. In the context of red blood cells, use of a non-toxic concentration of sugar is important because this enables direct transfusion of the red blood cells into a patient without first having to remove the cryoprotectant sugar from the red blood cells. In other words, use of a non-toxic sugar concentration enables direct, “one-step” transfusion of thawed blood.

As noted above, liposomes (liposomal vesicles) are used as a vector for transporting for intracellular delivery of non-permeant cryoprotective sugars such as disaccharides sucrose and trehalose. Development of this novel transport technique requires a consistent and well-characterized liposome product. Preferably, the liposomes that are used as delivery vehicles for loading sugar into the cells are manufactured to contain dipalmitoyl phosphatidylserine, dipalmitoyl phosphatidylcholine and cholesterol. It is also preferable that the liposomes are sized to have an outer diameter of 200 nm to 600 nm.

The liposomes (or “liposomal vesicles”) can be synthesized by lyophilization, hydration of dry lipid films, agitation to produce multilamellar vesicles, extrusion of the multilamellar vesicles to produce homogenous unilamellar vesicles with diameters in the range of 0.1-1 μm (and preferably 0.2-0.6 μm) depending on the extrusion filter pore size.

Liposomes having a lipid bilayer composed of 70% DPPC and 30% cholesterol provided excellent results in fusing with, and loading sugar into, red blood cells. However, it should be noted that the ratio of DPPC to cholesterol can, of course, be varied, as will be appreciated by those of ordinary skill in the art. The liposomes also have an aqueous core that can be composed of, by way of example only, 3.3 mM NaCl, 40 mM KCl, 1.7 mM glucose, 6.7 mM HEPES, 200 mM sucrose or trehalose and 100 μM 5(6)-carboxyfluorescein (CF). As it is known in the art, 5(6)-CF is an impermeable fluorescent marker with a molecular weight similar to that of sucrose and trehalose. This marker can be used to detect the liposome population using flow cytometry and is thus a useful and reliable indicator of loading efficiency. A single uniform population and intense FITC (Fluorescein isothiocyanate) peak verify liposome homogeneity and entrapment of the marker in the aqueous core.

Characterization of the final liposomal product is important for controlled, reproducible and effective liposomal delivery of cryoprotective intracellular disaccharides into mammalian cells for applications in biopreservation. A number of techniques are used to characterize the liposomes, including fluorometry which enables one to measure the total mean fluorescence of liposomes. Mean fluorescence intensity increases relative to the [5(6)-CF in the loading buffer, indicating controlled encapsulation of the dye.

Transmission electron microscopy can be also used to validate the size and to visually assess the morphology of the final liposome product. The size of the liposomes also can be determined using dynamic light scattering. In addition, a spectrophotometric assay of the phosphate content can estimate the total lipid concentration of the vesicle preparation, which allows for calculated and reproducible control of the liposome/cell ratio.

In order to validate the methods described herein, human red blood cells and TF-1 (CRL2003, ATTC) cells were used as nucleated and non-nucleated model systems. The mechanism of liposomal interaction with the cell is believed to be cell-type dependent and is believed to involve processes of adsorption, fusion and endocytosis.

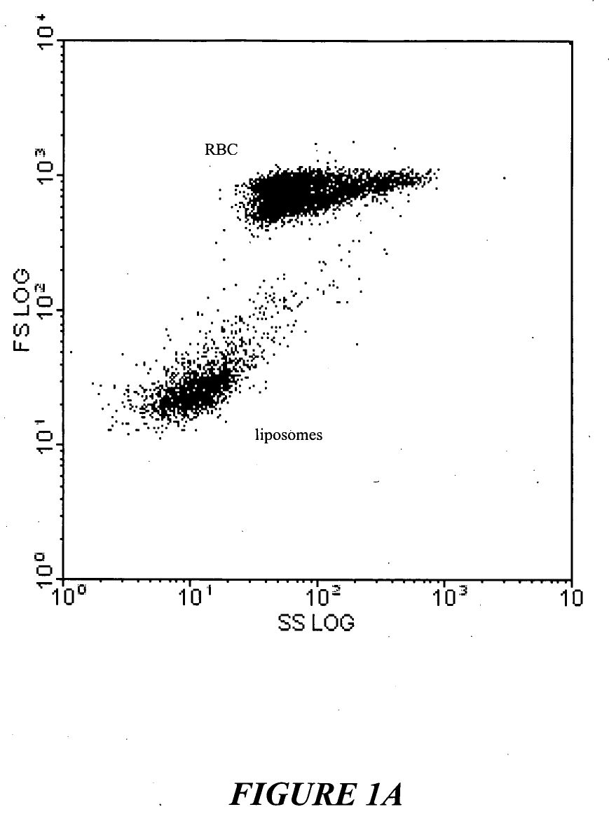

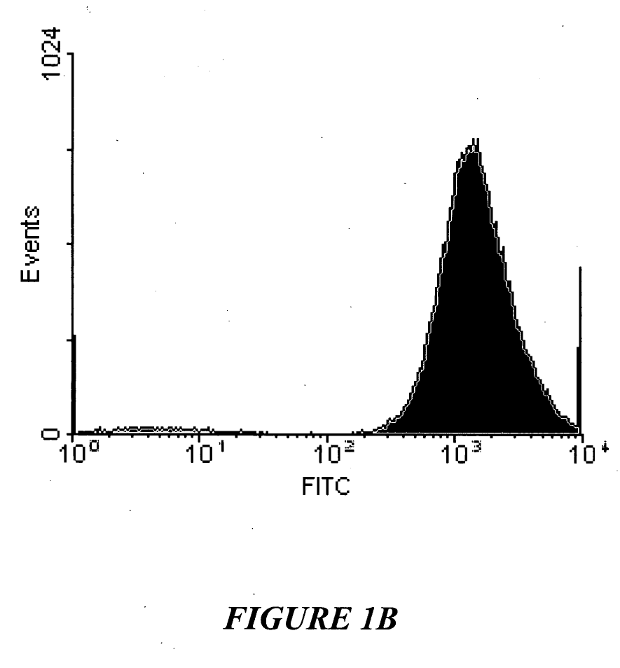

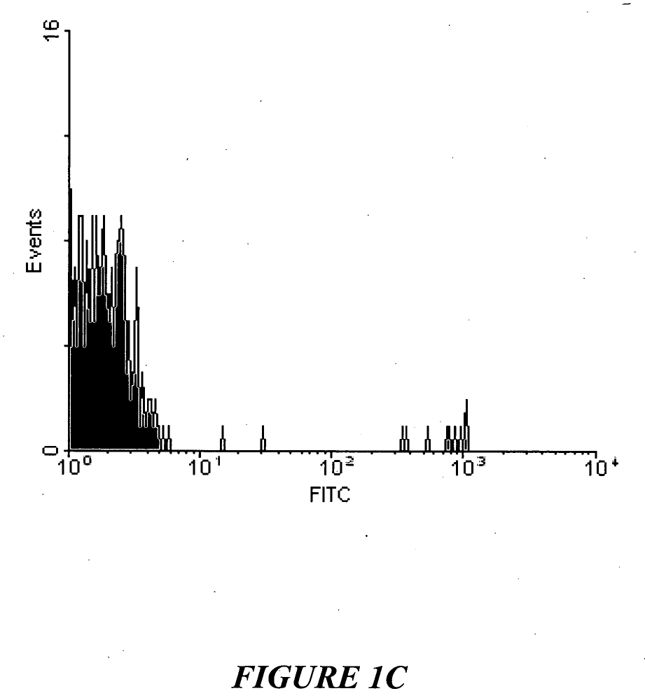

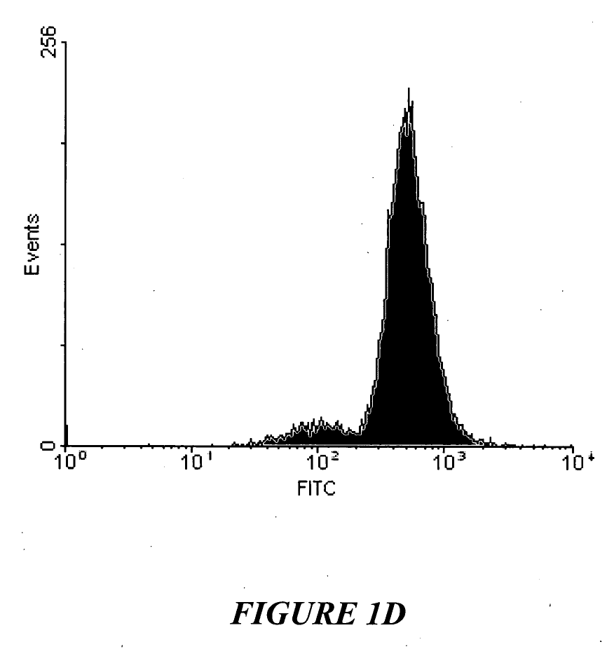

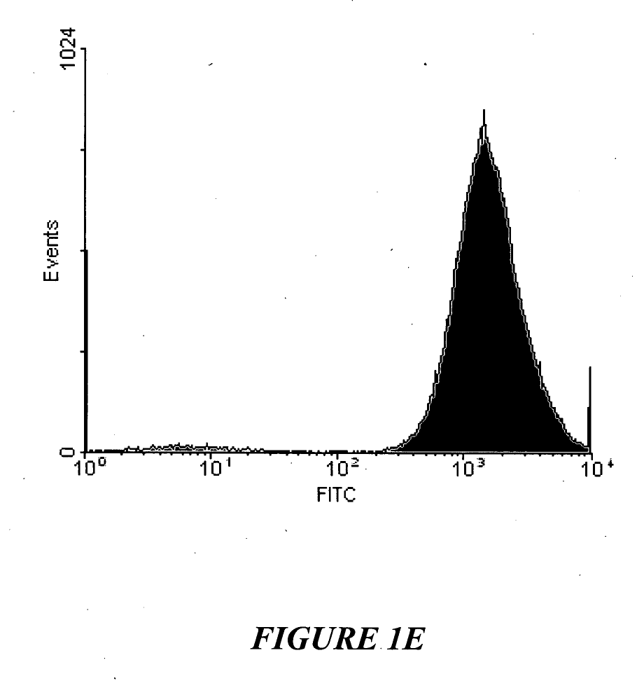

FIGS. 1A to 1E are flow cytometry plots used to examine the effects of loading trehalose into red blood cells (RBCs). Liposomes composed of DPPC/cholesterol in the ratio of 70:30 were constructed so as to have a concentration of 0.29M trehalose inside and outside. The additional presence of 0.1 mM carboxyfluorescein (CF) inside the liposomes was used to monitor movement of the liposome intracellular compartment. FIGS. 1C, 1D, 1E are gated on the RBC population.

FIG. 1A is a population distribution (“dot plot”) plotting the log of forward scatter (FS) versus the log of side scatter (SS). As forward scatter and side scatter define most cell populations, a population distribution such as the one shown in FIG. 1A can be used to differentially analyze RBCs and liposomes in a sample. Forward scatter is essentially an estimation of size, i.e. how long the laser beam is blocked as a cell passes in front of it, whereas side scatter is a measure of granularity/lumpiness, i.e. how much the laser beam is deflected by the unevenness of the cell passing in front of it.

FIG. 1B shows that the CF inside the liposomes is easily resolved. FIG. 1C shows that RBCs in CF-containing media are not fluorescent as CF cannot cross into RBCs. FIG. 1D shows the mixing of RBCs with CF/trehalose-loaded liposomes and storage overnight at 22° C. and movement of the CF from the liposomes into the RBCs. FIG. 1E shows how increasing the liposome/RBC ratio results in a significant increase of fluorescence within RBCs. Since the fusion of liposomes with RBCs is the mechanism by which sugars are delivered into mammalian cells, an increase of the liposome/RBC ratio improves delivery of sugar into the cells.

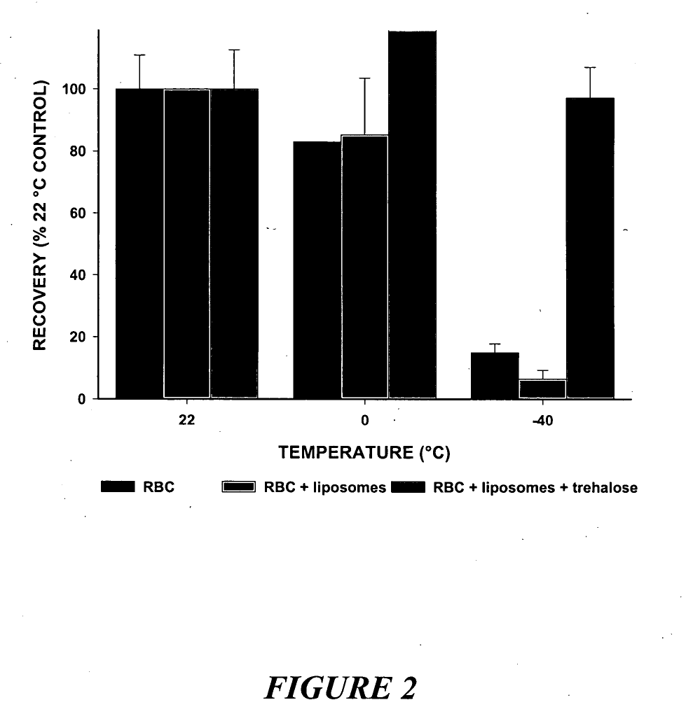

FIG. 2 is a bar graph plotting the recovery from 0° C. and from −40° C. of (i) red blood cells, (ii) red blood cells exposed to liposomes but containing no sugar and (iii) red blood cells exposed to liposomes containing 0.29M trehalose, as compared to 100% recovery from 22° C. (the control). FIG. 2 illustrates that red blood cells that are exposed to liposomes containing trehalose exhibit enhanced post-thaw viability, i.e. better “recovery”.

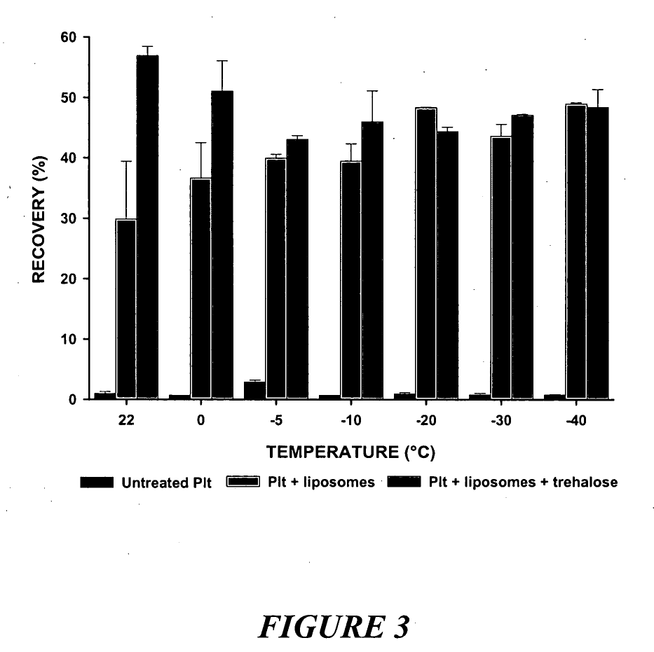

FIG. 3 is a bar graph plotting the recovery from 22° C., 0° C., −5° C., −10° C., −20° C., −30° C. and −40° C. of (i) human platelets in buffer, (ii) human platelets in buffer exposed to liposomes without sugar, and (iii) human platelets in buffer exposed to liposomes containing 0.29M trehalose. FIG. 3 illustrates that post-thaw viability of platelets is enhanced by exposing the platelets to trehalose-containing liposomes.

The methods described in the present application satisfy a long-felt need in the art of cryopreservation, particularly for clinical and commercial cell and tissue banking. Firstly, the methods of the present application enable a rapid, one-step addition of protective molecules into cells which the Applicant anticipates will replace the currently multi-step, gradient addition of permeant molecules currently being used to cryopreserve cells and tissues. This will greatly simplify the pre-processing requirements for preparing cells for cryopreservation.

Furthermore, as much lower concentrations of intracellular protectant are required to preserve cells during cryopreservation, the toxicity associated with the use of high concentrations of traditional cryoprotectants can be minimized or reduced. This would allow for a product that could be immediately transfused without first having to remove the cryoprotectant molecules.

Moreover, liposomes provide a means for the controlled delivery of a sufficient concentration of protectant molecules required to achieve cell cryopreservation. Other technologies are either not suited for use in clinical environments or do not result in the controlled delivery of significant concentrations of protective molecules. This will permit the cryopreservation of nucleated cells for use in commercial and clinical cell and tissue banking, an achievement that has yet to be accomplished by the prior art.

Finally, traditionally impermeable molecules having large molecular weight can now be used for the intracellular protection of cells during cryopreservation. The unique physicochemical properties of these molecules make them much better suited as protective molecules. The use of large molecular weight molecules is akin to the processes that natural systems (plants, animals, insects) use to survive extreme environmental stresses (i.e. winter). These molecules are more biocompatible than existing cryoprotectants and should cause fewer post-transfusion or post-transplantation complications.

In summary, and without limiting the foregoing, the liposomal delivery of intracellular sugar for the purposes of cryopreserving cells and tissues represents a substantial innovation over the prior-art methods of reversibly permeabilizing cells and/or accumulating protective molecules inside cells. Although the use of liposomes as transport vehicles is known in the art, the literature in the art has hitherto clearly suggested that liposomes would not function effectively to transport cryoprotectant sugars into cells. The methods of liposomal delivery of intracellular sugar that are described herein, however, represent a counterintuitive solution to the problems of enhancing post-thaw viability, in large measure due to the synergistic effects of the interactions of the liposome product and the cryoprotectant with the cell. Accordingly, a number of unexpected benefits arise from the methods described herein, the result of which are effective liposome-cell fusion, intracellular delivery of sugar and substantially improved post-thaw viability.

The embodiments of the present invention described above are intended to be exemplary only. The scope of the invention is therefore intended to be limited solely by the appended claims.

I/We claim:

1. A method for cryopreserving cells to enhance post-thaw cell viability, the method comprising steps of:
i) loading sugar into liposomes;
ii) causing the liposomes to interact with cell membranes of cells that are to be cryopreserved, thereby delivering the sugar as a cryoprotectant into the cells;
iii) cooling the cells to a predetermined nucleation temperature;
iv) nucleating extracellular ice; and
v) cooling the cells to a temperature lower than the predetermined nucleation temperature.

2. The method as claimed in claim 1 wherein the cells are mammalian.

3. The method as claimed in claim 1 wherein the cells are human.

4. The method as claimed in claim 1 wherein the cells are blood cells.

5. The method as claimed in claim 1 wherein the cells are erythrocytes.

6. The method as claimed in claim 1 wherein the cells are isolated cells in suspension.

7. The method as claimed in claim 1 wherein the cells are platelets.

8. The method as claimed in claim 1 wherein the cells are stem cells.

9. The method as claimed in claim 1 wherein the cells are components of a tissue.

10. The method as claimed in claim 1 wherein the cells are components of an organ.

11. The method as claimed in claim 1 wherein the sugar is selected from the group consisting of trehalose, sucrose, raffinose, and stachyose.

12. The method as claimed in claim 11 wherein the sugar has a concentration between 0.05M and 0.50M.

13. The method as claimed in claim 1 wherein the sugar comprises trehalose.

14. The method as claimed in claim 13 wherein the trehalose has a concentration of 0.29M.

15. The method as claimed in claim 1 wherein the sugar comprises a combination of at least two sugars selected from the group consisting of trehalose, sucrose, raffinose, and stachyose.

16. The method as claimed in claim 1 further comprising a prior step of manufacturing the liposomes to contain at least one of: dipalmityl phosphatidylserine, dipalmityl phosphatidylcholine, and cholesterol.

17. The method as claimed in claim 1 further comprising a prior step of sizing the liposomes to have an outer diameter of 200 to 600 nm.

18. The method as claimed in claim 16 further comprising a step of sizing the liposomes to have an outer diameter of 200 to 600 nm.

19. The method as claimed in claim 18 wherein the sugar is selected from the group consisting of trehalose, sucrose, raffinose, and stachyose.

20. The method as claimed in claim 19 wherein the sugar is dissolved in a solution having a physiologic osmolality, conductivity and pH.

21. A method of enhancing post-thaw viability of a cell to be cryopreserved, the method comprising steps of:
loading a cryoprotectant sugar into a liposome; and
causing the liposome to interact with the cell that is to be cryopreserved, the liposome delivering the cryoprotectant sugar into the cell for enhancing post-thaw viability of the cell.

22. The method as claimed in claim 21 further comprising a step of cryopreserving the cell containing the cryoprotectant sugar by cooling the cell below a nucleation temperature.

23. The method as claimed in claim 22 further comprising a step of thawing the cell by warming the cell above the nucleation temperature.

24. The method as claimed in claim 21 wherein the sugar is selected from the group consisting of trehalose, sucrose, raffinose, and stachyose.

25. The method as claimed in claim 22 wherein the sugar is selected from the group consisting of trehalose, sucrose, raffinose, and stachyose.

26. The method as claimed in claim 23 wherein the sugar is selected from the group consisting of trehalose, sucrose, raffinose, and stachyose.

27. The method as claimed in claim 21 wherein the sugar comprises a combination of at least two sugars selected from the group consisting of trehalose, sucrose, raffinose, and stachyose.

28. The method as claimed in claim 21 further comprising a prior step of manufacturing the liposomes to contain at least one of: dipalmityl phosphatidylserine, dipalmityl phosphatidylcholine, and cholesterol.

29. The method as claimed in claim 21 further comprising a prior step of sizing the liposomes to have an outer diameter of 200 nm to 600 nm.

30. The method as claimed in claim 21 wherein the cell is a red blood cell.

31. The method as claimed in claim 30 wherein a concentration of intracellular sugar delivered into the red blood cell is below a toxicity threshold, thereby enabling direct transfusion of the red blood cell without having to remove the cryoprotectant sugar from the red blood cell.

32. The method as claimed in claim 30 wherein a concentration of trehalose is between 0.20M and 0.30M.

33. The method as claimed in claim 30 wherein a concentration of trehalose is 0.29M.

34. The method as claimed in claim 21 wherein the liposome is composed of DPPC and cholesterol having a ratio of 70:30.

35. The method as claimed in claim 33 wherein the liposome is composed of DPPC and cholesterol having a ratio of 70:30.

36. The method as claimed in claim 21 wherein the sugar has a concentration of between 0.05M and 0.50M.
